(19) United States
(12) Patent Application Publication
(10) Pub. No.: US 2003/0143548 A1
(43) Pub. Date: Jul. 31, 2003

(54) Predicting patient responsiveness to serotonergic therapy

(76) Inventors: Michael L. Camilleri, Rochester, MN (US); Raul A. Urrutia, Rochester, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/058,630

(22) Filed: Jan. 28, 2002

(51) Int. Cl. C12Q 1/68; C12P 19/34

(52) U.S. Cl. 435/6; 435/91.2

(57) ABSTRACT

Methods to predict a patient's responsiveness to 5-HT₃ receptor antagonists are disclosed. The methods allow a clinician to predict a patient's responsiveness to 5-HT₃ receptor antagonists by determining the correlation that exists between a genotype in the promoter region of the gene encoding a serotonin transporter protein and patient response to 5-HT₃ receptor antagonist therapy. In addition, methods to treat patients suffering from diarrhea-predominant irritable bowel syndrome and methods to identify a patient population for inclusion in a 5-HT₃ receptor antagonist clinical trial are disclosed.

### STATEMENT AS TO FEDERALLY SPONSORED RESEARCH

Funding for the work described herein was provided in part by the Federal Government, which may have certain rights in the invention.

### TECHNICAL FIELD

This invention relates to serotonergic therapy, and more particularly to predicting a patient's responsiveness to serotonergic receptor antagonists.

### BACKGROUND

Irritable bowel syndrome (IBS) is a common gastrointestinal disorder in western populations, with an adult prevalence of approximately 15%. The cardinal features of IBS are recurrent abdominal pain and altered bowel habits. Diarrhea-predominant IBS has been associated with accelerated small bowel and/or colonic transit and with rectal hypersensitivity.

Serotonin (also known as 5-hydroxytryptamine or 5-HT) is an important gut neurotransmitter that modulates sensorimotor functions in the digestive tract. There are seven subclasses of serotonergic receptors, differentiated on the basis of structure, molecular mechanism, and function. Previous work has indicated that the 5-HT₃ class of serotonergic receptors are involved in postprandial colonic motor response and are distinct from the other six subclasses in that they are 5-HT ligand-gated ion channels, as compared to G protein-coupled receptors.

The endogenous inactivation of serotonin depends on a serotonin transporter protein that internalizes serotonin. This transporter protein is known to be central to the fine tuning of brain serotonin neurotransmission. The serotonin transporter protein is found in abundance in the cortical and limbic areas, which are areas involved in the emotional aspects of behavior and are hypothesized to malfunction in patients with irritable bowel syndrome. The same protein has been detected in the gastrointestinal tract in submucous and mysenteric ganglia as well as surface epithelial cells.

IBS has a strong female predominance of 70-75%. Clinical trials have documented a beneficial effect of alosetron, a 5-HT₃ receptor antagonist, in the adequate relief of IBS pain and discomfort and the normalization of bowel function in women with diarrhea-predominant IBS. Alosetron is able to slow colonic transit to a significantly greater degree among females than males with IBS. Although the pharmacokinetics of alosetron differ by gender, with slightly greater systemic exposure to alosetron in females compared to males, differences in pharmacokinetic profiles do not adequately explain the differences in efficacy between the sexes.

### SUMMARY

The invention is based on the discovery that the effectiveness of 5-HT₃ receptor antagonists on colonic transit may be related to the genotype in the promoter region of the gene encoding the serotonin transporter protein (5-HTTP). The promoter region of the 5-HTTP gene contains two polymorphic size variants: a long variant and a short variant. As described herein, a relationship exists between the long variant/long variant homozygous genotype in the promoter region of the 5-HTTP gene and greater patient responsiveness to 5-HT₃ receptor antagonists. Thus, the invention provides a predictive method for determining patient responsiveness to 5-HT₃ receptor antagonists for the treatment of diseases such as diarrhea predominant IBS and other related gastrointestinal disorders, such as functional or non-ulcer dyspepsia, vomiting syndromes, including cyclic vomiting, and other anti-emetic uses, such as ameliorating the side-effects of chemotherapy.

In one aspect, the invention features a method for predicting a patient's responsiveness to a 5-HT₃ receptor antagonist. The method includes determining the genotype of the promoter region of the patient's 5-HTTP gene and correlating the genotype with patient responsiveness to the 5-HT₃ receptor antagonist. The 5-HT₃ receptor antagonist can be used in a treatment for IBS and diarrhea-predominant IBS.

The 5-HT₃ receptor antagonist can be any one of a number of known compounds, including alosetron, ondansetron, granisetron, tropisetron, dolasetron, and cilansetron. Alosetron is particularly useful.

The genotyping step of the invention can include amplifying a nucleic acid that includes the promoter region of the patient's 5-HTTP gene to obtain an amplified product and determining the size of the amplified product to identify a long variant/long variant, short variant/long variant, or short variant/short variant genotype of the promoter region of the patient's 5-HTTP gene. Direct sequencing of the promoter region may also be performed to confirm the polymorphic variant size and promoter region locus. The genotyping step of the present invention thus determines whether the promoter region of the patient's 5-HTTP gene exhibits a long variant/long variant, short variant/long variant, or short variant/short variant genotype.

The correlating step of the present invention relates the particular genotype with patient responsiveness to the 5-HT₃ receptor antagonist. Patient responsiveness may be determined by measuring a patient parameter or by comparing a measured patient parameter with a pre-determined clinically significant threshold. In IBS, the measured patient parameter can be the change in the geometric center of colonic transit before and after treatment with the 5-HT₃ receptor antagonist. The pre-determined clinically significant threshold in IBS can be a net negative change in the geometric center of colonic transit of 1.14 colonic regions.

The presence of the long variant/long variant genotype in the promoter region of patient's 5-HTTP gene is related to greater patient responsiveness to a 5-HT₃ receptor antagonist, while the short variant/long variant genotype is not related to greater patient responsiveness to a 5-HT₃ receptor antagonist. Greater patient responsiveness to a 5-HT₃ receptor antagonist can result in a longer colonic transit period, particularly the achievement of the clinically significant threshold, or significant improvement in the patients' symptoms of IBS.

In another aspect, the invention features a method for treating a patient with diarrhea-predominant IBS that includes obtaining a biological sample, such as a blood, stool, or tissue (e.g. biopsy sample) sample; genotyping the promoter region of the sample's 5-HTTP gene; and administering to the patient an effective amount of a 5-HT₃ receptor antagonist if the patient has a long variant/long variant genotype in the promoter region of the 5-HTTP gene.

In yet another aspect, the invention includes a method for identifying a patient population for inclusion in a 5-HT₃ receptor antagonist clinical trial. The method includes obtaining a biological sample from a potential participant in the clinical trial; genotyping the promoter region of the biological sample's 5-HTTP gene; and identifying the potential participant as suitable for inclusion in the clinical trial based on the presence of a long variant/long variant genotype in the promoter region of the potential participant's 5-HTTP gene.

Unless otherwise defined, all technical and scientific terms used herein have the same meanings as commonly understood by one of skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A and FIG. 1B show a schematic and an electrophoretic separation, respectively, of the long and short variant polymorphisms in the serotonin transporter protein promoter region.

FIG. 2 demonstrates an example of the retardation of colonic transit in a patient after receiving alosetron 1 mg b.i.d ΔGC₂₄ₕ refers to the change in the colonic geometric center (weighted average location of counts in different colonic regions) 24 hours after treatment with alosetron.

### DETAILED DESCRIPTION

Phenotypic manifestations of diarrhea-predominant IBS may be partly influenced by the neurotransmitter serotonin, since circulating plasma postprandial levels of serotonin (5-HT) are elevated. Mucosal biopsies in a subset of patients with post-infectious IBS also show increased numbers of enteroendocrine cells containing serotonin. Moreover, pharmacological and clinical studies have suggested that agents that block the effects of endogenous serotonin at the receptor, particularly the 5-HT₃ receptor subclass, are effective in treatment of a subset of IBS patients (Steadman C. J. et al., Mayo Clin. Proc. 67:732-38 (1992); Prior A. and Read N. W., Aliment. Pharm. Ther. 7:175-180 (1993); and Camilleri M. et al., Lancet 355:1035-40 (2000)).

Colonic transit has been shown to be significantly prolonged by alosetron, a 5-HT₃ receptor antagonist, in patients with diarrhea-predominant IBS, with greater effect in females than males. However, a statistically significant response of a similar magnitude in some male patients has also been recognized, indicating that factors other than gender may be responsible for the greater response.

The endogenous inactivation of serotonin depends on 5-HTTP, which internalizes serotonin. The present invention is based on the surprising discovery that patient responsiveness to 5-HT₃ receptor antagonists can be correlated with a particular genotype in the promoter region of the 5-HTTP gene. As defined herein, genotype means the polymorphic size variant for each of the two alleles of the promoter region of the 5-HTTP gene. See, GenBank Accession No. X76753 for the nucleotide sequence of the polymorphic region of the human 5-HTTP gene. The polymorphic size variant can be a long variant/long variant, short variant/long variant, or short variant/long variant. Heils et al., J. Neurochem. 66:2621-2624(1996). In the long variant, there is a 44 bp insertion, and in the short variant, there is a 44 bp deletion. Thus, a clinician may predict a patient's responsiveness to 5-HT₃ receptor antagonists by determining the genotype in the promoter region of the 5-HTTP gene and correlating the genotype with the response to 5-HT₃ receptor antagonist therapy.

As described herein, patients having the long variant/long variant have greater responsiveness to 5-HT₃ receptor antagonists. Such a discovery allows the clinician to predict a patient's responsiveness to 5-HT₃ receptor antagonists in the treatment of disease such as diarrhea-predominant IBS. It further facilitates the identification and treatment of patients that exhibit a genotype that is correlated with greater responsiveness to 5-HT₃ receptor antagonists. Finally, the discovery allows tailoring of patient populations in clinical trials by identifying those patients more likely to demonstrate greater responsiveness to a 5-HT₃ receptor antagonist based on the patient's genotype in the promoter region of the 5-HTTP gene.

The 5-HT₃ receptor antagonist can be used in a treatment for IBS and diarrhea-predominant IBS, as well as for antiemitic purposes. The 5-HT₃ receptor antagonist can be any one of a number of known compounds, including alosetron (Lotronex™, Glaxo-Wellcome Pharmaceuticals), ondansetron (Zofran™, Glaxo-Wellcome Pharmaceuticals), granisetron (Kytril™, SmithKline Beecham), tropisetron (Novoban), dolasetron (Anzemet™, Hoescht Marion Roussel, Inc.), and Cilansetron (Solvay Pharmaceuticals). Of course, newly discovered 5-HT₃ receptor antagonists and compounds under investigation as 5-HT₃ receptor antagonists are also contemplated for use in the present invention.

Genotyping the Promoter Region of the Gene Encoding 5-HTTP

In general, a biological sample is obtained from the patient, such as a blood, tissue (e.g., a biopsy), oral washings, or stool sample. Blood is a particularly useful biological sample. Genomic DNA is then extracted from the biological sample. Routine methods can be used to extract genomic DNA from biological samples, including, for example, phenol extraction. Alternatively, genomic DNA can be extracted with kits such as the QIAamp® Tissue Kit (Qiagen, Chatsworth, Calif.), Wizard® Genomic DNA purification kit (Promega, Madison, Wis.), and the A.S.A.P. Genomic DNA isolation kit (Boehringer Mannheim, Indianapolis, Ind.).

Typically an amplification step is performed before proceeding with the determination of the size variant in the promoter region. Polymerase chain reaction (PCR) methods can be used to obtain an amplified product that includes the promoter region of the 5-HTTP gene to be genotyped in the present invention. Since the nucleotide sequence of the regions flanking the two polymorphic variants of the 5-HTTP gene promoter region are known, PCR primers that are identical in sequence to the opposite strands of the template to be amplified can be designed, synthesized, and used to amplify the promoter region. PCR primers are typically 14 to 40 nucleotides in length, but can range from 10 nucleotides to hundreds of nucleotides in length. General PCR techniques are described, for example, in PCR Primer: A Laboratory Manual, Ed. By Dieffenbach, C. and Dveksler, G., Cold Spring Harbor Laboratory Press, 1995. See, Example 3 for the sequence of particular primers that can be used.

The amplified product can be separated by size electrophoretically and compared with size standards in order to determine the length of the polymorphic size variant. The amplified products can be electrophoresed, for example, through agarose or acrylamide gels, usually at a concentration of between about 1% to about 20% (e.g., 1 to 4% agarose), and compared to a set of size standards. Standard gel electrophoresis techniques are described in Molecular Cloning, A Laboratory Manual, 2d Edition, J. Sambrook, E. F. Fritsch, and T. Maniatis, Eds., Cold Spring Harbor Press, Vol. 1, Ch. 6 (1989). When using the primers described herein, an amplified product containing the long variant/long variant is approximately 572 nucleotides in length, while the product containing the short variant is 528 nucleotides in length. Standard techniques can be used to visualize the DNA, including the use of ethidium bromide or other DNA intercalating dye visible under UV light. Alternatively, the amplified DNA can be detected by labeling a PCR primer with a fluorescent moiety, while the size standards may be labeled with a different fluorescent moiety.

Direct sequencing of the promoter region of the 5-HTTP gene may also be performed using standard techniques. Standard DNA sequencing techniques are described in Molecular Cloning, A Laboratory Manual, 2d Edition, J. Sambrook, E. F. Fritsch, and T. Maniatis, Eds., Cold Spring Harbor Press, Vol. 2, Ch. 13 (1989).

Correlating Genotype With Patient Responsiveness

The patient's genotype in the promoter region of the 5-HTTP gene (long variant/long variant, short variant/long variant, or short variant/short variant genotype) can be correlated with responsiveness to a 5-HT₃ antagonist. Standard statistical techniques may be used to determine if a relationship exists between any of the three genotypes and a change in a measured patient parameter, i.e., any measurable manifestation of the disease pathophysiology. In diarrhea-predominant IBS, the measured patient parameter can be the geometric center of colonic transit and the change in the geometric center of colonic transit post treatment. In some embodiments, the change in the measured patient parameter can be compared with a pre-determined clinically significant threshold after treatment with a 5-HT3 receptor antagonist. The pre-determined clinically significant threshold is often related to a beneficial change in some manifestation of the disease. A predetermined clinically significant threshold may be pre-determined by, for example, a clinician familiar with the pathologic expression of the disease.

The measured patient parameter can be examined both before and after treatment with a is 5-HT₃ receptor antagonist. For example, in IBS, the geometric center of colonic transit can be examined for a period of time (e.g., 12 hours, 24 hours, 36 hours) before and after treatment with a 5-HT3 receptor antagonist, e.g., alosetron. The net change in the measured patient parameter can then be calculated and can subsequently represent the measured patient parameter.

The pre-determined clinically significant threshold can be related to the measured patient parameter. In 39 healthy subjects, the colonic transit measured as the geometric center at 24 hours was 2.7±0.18 (SEM). In other studies, the geometric center at 24 hours was 1.8±0.2 in severe idiopathic constipation (see Stivland et al. Gastroenterology 1991;101:107-15.), 3.3±0.35 in diarrhea-predominant IBS (Vassallo et al. Gastroenterology 1992;102:102-8.), and 4.5±0.4 in carcinoid diarrhea (von der Ohe et al. N Engl J Med 1993;329:1073-8). The average difference in colonic geometric center at 24 hours for disease states relative to controls is 1.1 ([0.9+0.6+1.8]/3=1.1). Thus, in diarrhea-predominant IBS, a change of 1.14 units in the geometric center of colonic transit at 24 hours from baseline can be deemed a priori as a clinically significant alteration of transit. Alternatively, a clinically significant threshold may be pre-determined to be a defined change in geometric center of colonic transit at a lower dosage of the particular 5-HT₃ receptor antagonist.

The patient responsiveness to a 5-HT₃ receptor antagonist may be determined by comparing the pre-determined clinically significant threshold with a measured patient parameter before and after treatment. For example, if the predetermined clinically significant threshold is a change of 1.14 units in the geometric center of colonic transit, the measured patient parameter to be compared is the net change in the geometric center of colonic transit for a set period of time, e.g., 24 h. before and after treatment with a 5-HT₃ receptor antagonist.

Colonic transit can be monitored by known methods (Camilleri M. and Zinsmeister A. R., Gastroenterology 103:36-42 (1992); and Camilleri M. et al., Dig. Dis. Sci. 36:609-615 (1991)). In general, labeled (e.g., ¹¹¹In) pellets can be delivered to the colon by means of a methacrylate-coated, delayed-release capsule. Simultaneously, labeled (e.g., ^{99m}Tc) pellets can be ingested in a scrambled egg, toast, and milk meal to facilitate measurement of gastric and small bowel transit. A large field gamma camera (e.g., GE 500, General Electric, Milwaukee, Wis.) with a medium energy parallel hole collimator can be used to obtain images. Typically, images are obtained with subjects in the erect position. A variable region of interest (ROI) program can be used to measure colonic transit, with abdominal images obtained at periodic intervals (e.g.,4, 6, 8, 24, 32, and 48 hours pre- or post-treatment. The amount of label can be determined from the abdominal images in four colonic regions: the ascending, transverse, descending, and combined sigmoid and rectum regions. These counts may be corrected for isotope decay and tissue attenuation, as described in Camilleri et al. (1991) and Camilleri and Zinsmeister (1992), referenced above. The colonic geometric center is the weighted average of counts in the four different colonic regions [ascending (AC), transverse (TC), descending (DC), rectosigmoid (RS) and stool]:

(% AC×1+% TC×2+% DC×3+% RS×4+% stool×5)/100=geometric center

A high geometric center implies faster colonic transit, while a reduction of geometric center with treatment implies a retardation of colonic transit.

Known statistical methods may be used to correlate the measured patient parameter or the comparison of the pre-determined clinically significant threshold with the measured patient parameter with a particular genotype in the promoter region of the 5-HTTP gene. For example, analysis of variance (ANOVA), Mann-Whitney rank sum tests, and Fisher's Exact test methods may all be employed.

In the case of treatment with a 5-HT₃ receptor antagonist, the presence of a particular genotype in a patient's promoter region of the 5-HTTP gene may be indicative of greater or lesser patient responsiveness. For example, greater patient responsiveness to a 5-HT₃ receptor antagonist can be manifested by a reduction in the measured patient parameter, such as, in IBS, a net reduction in the geometric center of colonic transit post treatment and thus a longer colonic transit period. Greater patient responsiveness can also be manifested by the achievement of the pre-determined clinically significant threshold. In diarrhea-predominant IBS, greater patient responsiveness may be demonstrated by, for example, a net negative change of at least about 1.14 colonic regions in the colonic geometric center at 24 hr. post treatment with the 5-HT₃ receptor antagonist. A net negative change in the colonic geometric center at 24 hr. is indicative of slower transit through the colon. In the present invention, the long variant/long variant genotype is related to a greater patient responsiveness to the 5-HT₃ receptor antagonist than the heterozygous long variant/short variant genotype in the treatment of diarrhea-induced IBS.

Methods of Treatment

In another aspect, the invention features a method for treating a patient with diarrhea-predominant IBS. A biological sample can be obtained from the patient and the genotype of the promoter region of the 5-HTTP gene can be determined as described above. Patients having the long variant/long variant genotype are administered an effective amount of a 5-HT₃ receptor antagonist. Effective amounts of 5-HT₃ receptor antagonists used in the present invention will depend on many factors including the mode of administration, the severity of the IBS disease, and the pharmacodynamic and pharmacokinetic profile of the particular formulation in vivo. Typically, 5-HT₃ receptor antagonists dosages range from about 0.05 to 20 mg once or twice daily. For example, 0.5 to 4 mg of alosetron can be administered once or twice daily. Generally, the amount administered will be a dosage that effectively engenders a beneficial patient response without inducing significant toxicity.

The 5-HT₃ receptor antagonists can be administered by any suitable route, including enteral (e.g., oral) and parenteral (e.g., intravenous, subcutaneous, or intramuscular). A preferred route of administration can depend on a variety of factors, such as disease, pharmacokinetic factors, clinician judgment, and therapeutic goals. Oral administration is particularly useful.

Typically, a 5-HT₃ receptor antagonist is mixed with a pharmaceutically acceptable carrier or excipient. Various pharmaceutically acceptable carriers can be used, including for example physiological saline or other known carriers appropriate to specific routes of administration. Preparations for administration can include sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water as well as alcohol, saline, and buffered solutions. Preservatives, flavorings, and other additives such as, for example, antimicrobials, anti-oxidants, chelating agents, inert gases, and the like may also be present.

Tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). The tablets can be coated by methods known in the art.

Liquid preparations for oral administration can take the form of, for example, solutions, syrups or suspension, or they can be presented as a dry product for constitution with saline or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitable formulated to give controlled release of the compound, including for example the use of liposomal or microsphere formulations.

Identifying Patients For Clinical Trials

The invention also includes a method for identifying a suitable patient population for a 5-HT₃ receptor antagonist clinical trial. Biological samples (e.g., a blood, stool, or tissue sample) can be obtained from a potential participant in a clinical trial and the genotype of the promoter region of the 5-HTTP gene can be determined as described above. Patients having the long variant/long variant genotype are suitable for inclusion in the clinical trial. One of skill in the art will recognize that possession of the long variant/long variant genotype is not sufficient for certain inclusion in the clinical trial, as other factors impact the decision to enroll a patient, including the patient's general health, prior use of similar agents in the treatment of the disease, clinical endpoints to be monitored, and the like. However, possession of the long variant/long variant genotype has been shown in the present invention to correlate with higher patient responsiveness to the 5-HT₃ receptor antagonists, and thus provides the clinical trial coordinator with a simple and cost-effective method to pre-screen potential participants. Such a method to identify a patient population for possible inclusion in a clinical trial for 5-HT₃ receptor antagonists can improve the approval rate of these drugs at, for example, the FDA, and provide a more appropriate population on which to study their efficacy and safety.

The invention will be further described in the following examples, which do not limit the scope of the invention described in these claims.

### EXAMPLES

### Example 1

Patient Population and Timing of Physiologic Studies

Thirty patients with diarrhea-predominant IBS associated were studied. Gastrointestinal and colonic transit were performed during a baseline period and during the last week of a 6-week therapeutic trial with alosetron 1 mg twice daily (b.i.d.). Demographic data of the original 30 participants are as follows: mean age 43.2±3y (females 46±4y, males 40±4y); mean duration of IBS 11.2±2.0y (females 9.2±2.3y and males 13.1±3.2y). Twenty-three of the 30 patients agreed to participate in an evaluation of the influence of the genotype of the promoter region of the 5-HTTP gene. The demographics, gender, and duration of illness in the three groups of patients according to 5-HTTP genotype are shown in the table below.

| | Long | Short | | |
|---|---|---|---|---|
| | Homozygous | Homozygous | Heterozygous | P value |
| N | 8 | 4 | 11 | |
| Gender (M:F) | 2:6 | 2:2 | 7:4 | 0.25 |
| Age (y) | 39.3 ± 5.3 | 45.8 ± 6.6 | 47.5 ± 5.0 | 0.52 |
| Duration of | 113 ± 54 | 117 ± 52 | 142 ± 37 | 0.88 |
| IBS (months) | | | | |

Informed consent and peripheral blood DNA samples were obtained from the 23 patients using the alkaline lysis method using the QIAmp DNA Blood Maxi Kit (Qiagen Inc., Valencia, Calif.).

### Example 2

Measurement of Colonic Transit

An established scintigraphic method was used as referenced above. Briefly, ¹¹¹In pellets were delivered to the colon by means of a methacrylate-coated, delayed-release capsule. Simultaneously, ^{99m}Tc pellets were ingested in a scrambled egg, toast, and milk meal (218 kcal) to facilitate measurement of gastric and small bowel transit. Subjects ingested standardized meals for lunch and dinner. A variable region of interest (ROI) program was used to measure colonic transit. Abdominal images at 4, 6, 8, 24, 32, and 48 hours were obtained using a large field of view gamma camera (GE 500, General Electric, Milwaukee, Wis.) with a medium energy parallel hole collimator. All images were obtained with the subjects in the erect position. Radioscintigraphic markers were placed for reference on the anterior ends of the iliac crests. Anterior and posterior images were obtained with the camera positioned over the abdomen. This facilitated subsequent correction of radioisotopic counts for tissue attenuation.

The counts in four colonic regions were determined: the ascending, transverse, descending, and combined sigmoid and rectum regions. These counts were corrected for isotope decay and tissue attenuation, as referenced above. The primary endpoint of investigation was the colonic geometric center at 24 hours. The colonic geometric center is the weighted average of counts in the four different colonic regions [ascending (AC), transverse (TC), descending (DC), rectosigmoid (RS) and stool]:

(% AC×1+% TC×2+% DC×3+% RS×4+% stool×5)/100=geometric center

Thus, a high geometric center implies faster colonic transit, while a reduction of geometric center with treatment implies a retardation of colonic transit.

### Example 3

Analysis of the 5-HTTP Gene Promoter Region Polymorphic Size Variant Genotypes

Polymorphic regions in the 5-HTTP gene (GenBank Accession No. X76753) were amplified by polymerase chain reaction using the following primers:

5′-GGAGGAACTGACCCCTGAAAACTG-3′ and

5′-GCCGCTCTGAATGCCAGCAC-3′, which flank the 5-HTTP long polymorphic region and correspond with nucleotide positions 2219 to 2242 and 1671 to 1680 of the 5-HTTP gene, respectively.

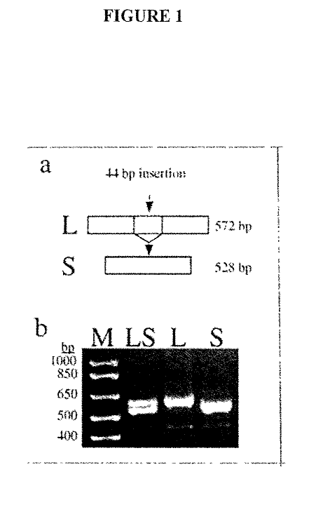

Reactions contained 0.7 mg genomic DNA, 400 mM deoxyribonucleotides, and 0.2 mM of each primer in a 50 μl reaction volume. Because of the high guanine and cytosine (GC) content in this region of the gene, PCR was performed using the TaKaRa La Taq polymerase (2.5U/reaction) with GC Buffer I, (TaKaRa Biomedicals, Shiga, Japan). After denaturing the sample at 94° C. for 1 minute, cycling conditions were set at 30 cycles of 94° C. for 30 sec, 60° C. for 30 sec, 72° C. for 2 min. Amplified products were electrophoresed through 1.5% agarose to determine the presence of length variations of the alleles. Genotypes were confirmed by direct sequencing at the Mayo Gene Sequencing Core Facility, using an ABI Prism system. FIG. 1A contains a schematic of the amplified products.

### Example 4

Statistical Analysis

Changes in colonic transit between baseline and 6 weeks' post-alosetron treatment in the three genotypic groups of patients were compared by a non-parametric analysis of variation (ANOVA), as the transit results were not normally distributed; two group non-parametric comparisons followed using the Mann-Whitney test. The proportion of clinically meaningful responders in the different genotype groups was determined using Fisher's exact test. The α level for statistical significance was set at 0.05. All analyses were performed using SigmaStat Statistical Software Version 2.0 for Windows, NT and 3.1(SPSS Inc. Chicago, Ill.).

### Example 5

Effect of Alosetron on Colonic Transit

The colonic geometric centers at 24 hours were significantly lower (suggesting slower overall colonic transit) in the group of patients with IBS following treatment with alosetron compared to baseline.

The change in overall colonic transit was significantly greater in females compared to males, with a change (delta, Δ) in colonic geometric center (ΔGC) at 24 hr of −1.45±0.25 in females, and −0.32±0.27 in males, and a p=0.005. Note that a negative sign in the colonic geometric center implies slower transit through the colon.

Studies of the individual transit profiles showed that 2 of 15 males had a retardation of colonic transit at 24 hours that was equal to or greater than the mean change (1.45 geometric center units) in females observed in this study. Nine females and 3 males had a retardation of colonic transit that was greater than the predetermined clinically significant threshold of 1.14 colonic regions, suggesting that the alosetron affected colonic function in these individuals to a degree that achieved a clinically meaningful difference. In 2 males, slowing of colonic transit was greater than the mean change in females. Conversely, alosetron had no effect on colonic transit in 2 females.

### Example 6

Serotonin Transporter Protein Promoter Region Polymorphic Size Variant Genotypes and Relationship to Change in Colonic Transit

FIG. 1B shows an electrophoretic separation of the long and short variant polymorphisms in the promoter region of the 5-HTTP gene. Four patients demonstrated a short variant/short variant homozygous genotype, and eight patients demonstrated a long variant/long variant homozygous genotype in the promoter region. The remaining 11 patients were heterozygous with one short and one long variant allele.

Polymorphisms within the 5-HTTP promoter region tended to be associated with colonic transit response (ANOVA on ranks, p=0.075), with significantly higher response in long variant/long variant homozygous than long variant/short variant heterozygous patients (p=0.039), but not between short variant/short variant homozygous and long variant/short variant heterozygous patients (p=0. 17).

The pre-determined clinically significant threshold of slowing of colonic transit by more than 1.14-regions was significantly more frequent with the long variant/long variant homozygous genotype than in the remaining patients, who were either heterozygous or had a homozygous short variant polymorphism (Fisher's exact test, p=0.035). The proportion of high responders was 6 of 8 in the long variant homozygous group, and 2 of 11 in the heterozygous group (Fisher's exact test, p=0.024).

Age, gender and duration of IBS were not different in the three groups of patients classified by the type of polymorphism.

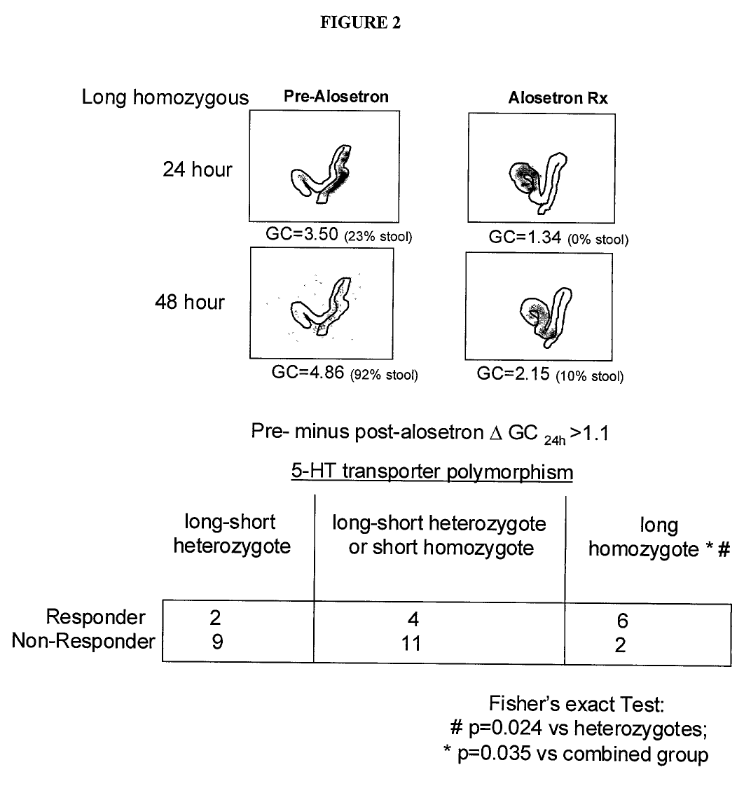

FIG. 2 demonstrates the results of patient groups with polymorphic size variant genotype in the promoter region of the 5-HTTP gene and the predetermined clinically significant threshold change in colonic transit of 1.14 units after treatment with alosetron. ΔGC₂₄ₕ refers to the change in the colonic geometric center 24 hours after treatment with alosetron. The colonic geometric center was taken as the weighted average of scintigraphic counts in four colonic regions. In general, there is a correlation between polymorphic size variant genotype and the change in colonic transit of 1.14 units after treatment with alosetron such that a greater response was observed in those with the long polymorphism.

Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method for predicting patient responsiveness to a 5-HT₃ receptor antagonist, said method comprising:
(a) determining genotype of the promoter region of said patient's 5-HTTP gene; and
(b) correlating said genotype with patient responsiveness.

2. The method of claim 1, wherein said 5-HT₃ receptor antagonist is used in a treatment for diarrhea-predominant irritable bowel syndrome.

3. The method of claim 1, wherein said 5-HT₃ receptor antagonist is selected from the group consisting of: alosetron, ondansetron, granisetron, tropisetron, and dolasetron.

4. The method of claim 1, wherein said 5-HT₃ receptor antagonist is alosetron.

5. The method of claim 1, wherein said genotyping step comprises:
(a) amplifying a nucleic acid comprising the promoter region of said patient's 5-HTTP gene to obtain an amplified product; and
(b) determining the size of said amplified product to identify a long variant/long variant, short variant/long variant, or short variant/short variant genotype of the promoter region of said patient's 5-HTTP gene.

6. The method of claim 5, wherein said correlating step comprises relating said long variant/long variant, short variant/long variant, or short variant/short variant genotype with patient responsiveness.

7. The method of claim 6, wherein said long variant/long variant genotype is related to a greater patient responsiveness than said short variant/long variant genotype.

8. The method of claim 6, wherein said patient responsiveness is determined by measuring a patient parameter.

9. The method of claim 6, wherein said patient responsiveness is determined by comparing a measured patient parameter with a pre-determined clinically significant threshold.

10. The method of claim 9, wherein said measured patient parameter is a net negative change in a geometric center of colonic transit after treatment with said 5-HT₃ receptor antagonist.

11. The method of claim 9, wherein said pre-determined clinically significant threshold is a net negative change in the geometric center of colonic transit of at least about 1.14 colonic regions.

12. A method for treating a patient with diarrhea-predominant irritable bowel syndrome comprising:
(a) obtaining a biological sample from said patient;
(b) genotyping the promoter region of said sample's 5-HTTP gene; and
(c) administering to said patient an effective amount of a 5-HT₃ receptor antagonist if said patient has a long variant/long variant genotype in the promoter region of the 5-HTTP gene.

13. The method of claim 12, wherein said biological sample is selected from the group consisting of a blood and a tissue sample.

14. A method for identifying a patient population for inclusion in a 5-HT₃ receptor antagonist clinical trial comprising:
(a) obtaining a biological sample from a potential participant in said clinical trial;
(b) genotyping the promoter region of the 5-HTTP gene contained within said biological sample; and
(c) identifying said potential participant as suitable for inclusion in said patient population based on the presence of a long variant/long variant genotype in the promoter region of said potential participant's 5-HTTP gene.
